(19) United States
(12) Patent Application Publication
(10) Pub. No.: US 2003/0036518 A1
(43) Pub. Date: Feb. 20, 2003

(54) ARTIFICIAL POLYMERIC MEMBRANE STRUCTURE, METHOD FOR PREPARING SAME, METHOD FOR PREPARING THIS POLYMER, PARTICLE AND FILM CONTAINING THIS STRUCTURE

(76) Inventors: DANIEL SAMAIN, TOULOUSE (FR); ETIENNE PEROCHON, TOULOUSE (FR)

Correspondence Address: YOUNG & THOMPSON, 745 SOUTH 23RD STREET, SECOND FLOOR, ARLINGTON, VA, 22202

(21) Appl. No.: 09/284,754

(22) PCT Filed: Oct. 22, 1997
(86) PCT No.: PCT/FR97/01891

(30) Foreign Application Priority Data
Oct. 23, 1996 (FR) 9613101

(51) Int. Cl. A61K 48/00; A61K 9/70

(52) U.S. Cl. 514/44; 424/443; 424/490

(57) ABSTRACT

The invention concerns an artificial membranous structure analogous to fixed plasmic membranes which comprise a solid substrate (61); a functional membrane (63) which does not circumscribe the external medium; and at least one bifunctional fixing compound (62) inserted between the membrane (63) and the substrate (61), cooperating by polyelectrolytic complexing with the substrate (61) and by lyotropic bonds with the membrane (63). The invention also concerns the use of this structure for obtaining a medicament, a particle, a film, its method of preparation, as well as a lipidic polycationic polymer, and a method for its preparation, acting as a bifunctional compound (62).

The invention concerns an artificial membranous structure analogous to natural plasmic membranes fixed to a cytoskeleton, such as are found in eucaryote cells, in particular in erythrocytes and derivatives of eucaryote cells which constitute enveloped viruses.

Natural plasmic membranes (cf. for example MOLECULAR BIOLOGY OF THE CELL, Third edition, 1994, Bruce Alberts et al., Garland Publishing, New York, USA, or THE MEMBRANES OF CELLS, Philip YEAGLE, 1987, Academic Press Inc., San Diego, USA) comprise a lipidic (mainly phospholipidic) bilayer forming a stable lamellar lyotropic phase incorporating amphiphilic molecules and proteins held together by non-covalent stable lyotropic interactions (mainly involving so-called “hydrophobic” bonds). The amphiphilic molecules are arranged in a continuous double layer having a thickness of the order of 4 to 5 nm in the liquid crystal and fluid mosaic state (cf. Singer and Nicholson, Science, Vol. 175, 720 (1972)) which acts as a semipermeable barrier to most of the large neutral polar molecules, among these being sugars, and to inorganic ions, while being permeable to hydrophobic molecules (in particular oxygen) and to small neutral polar molecules (in particular water, urea and glycerol). The transmembranous proteins of natural membranes enable, in particular, ions and sugars etc to be transported selectively through the bilayer. Natural plasmic membranes also carry systems governing exchanges with the outside (in general proteins or sugars).

The natural plasmic membranes of eucaryote cells are fixed to a cytoskeleton (network of filamentary proteins) which stabilizes the plasmic membrane from the inside. Fixing involves an assembly of specific bonds between the proteins anchored in the membrane and proteins of the cytoskeleton.

Thus, the envelopes of enveloped viruses are formed of a plasmic membrane fixed to the capsid of the virus by specific proteins of the viral envelope capable of bonding with the proteins of the capsid. Equally, the membrane of erythrocytes is fixed to a fibrous cytoskeleton by means of a band 3 protein, capable of bonding with the ankyrin of the cytoskeleton, which gives it a biconcave form (which is transformed into a spherocyte when fixing is deficient).

Accordingly, fixed plasmic membranes not only possess the fundamental properties specific to lipidic bilayers, but they may also exhibit a complex morphology (in particular with a considerable surface area, for example intestinal cells provided with microvilli, or the axones of neurones) and possess, on account of the fixing of the bilayer onto the cytoskeleton, remarkable mechanical properties, in particular strength and stability. Moreover, the fixing system preserves the lateral fluidity of the bilayer and the fixed membranes retain the property of being autosealable, as are liposomes, i.e. they are capable of closing spontaneously, in particular after having experienced perforation.

Taking into account the importance of these properties, specific to fixed plasmic membranes, it would be highly desirable to be able to obtain analogous artificial structures.

Now, although it has been known for a long time how to produce artificial structures formed of a lipidic bilayer (for example liposomes), it has not been found possible up to now to prepare, in a simple manner compatible with industrial constraints, artificial membranous structures imitating the natural plasmic membranes of fixed eucaryote cells. Indeed, the cytoskeleton, proteins enabling the bilayer to be fixed to the cytoskeleton, and specific bonds between these proteins and the cytoskeleton, are extremely complex to manipulate and to use artificially.

Thus, the publication “Molecular Architecture and Function of Polymeric Oriented Systems: Models for the Study of Organization, Surface Recognition and Dynamics of Biomembranes”, Helmut Ringsdorf et al., Angew. Chem. Int. Ed. Engl. 27 (1988) 113-158 describes known artificial supramolecular systems and indicates the methods that could be considered for simulating a membrane fixed to a cytoskeleton (§4.8.215 p 136-138):

1—associating, by means of direct covalent bonds, networks of linear polymers acting as a skeleton with one of the layers of the lipidic bilayer.

2—filling the interior of a liposome with a polymer gel forming a three-dimensional network which is not bonded to the lipidic bilayer,

3—associating, by means of direct electrostatic interactions, networks of linear polyionic polymers acting as a skeleton with one of the layers of the lipidic bilayer of which the polar heads are also ionic.

The first method only enables stabilized spherical polymerized liposomes to be obtained, and does not allow a membranous structure to be produced which has any shape and which is autosealable. Moreover, the bilayer has polymerized portions in which lateral fluidity is absent, and unpolymerized portions which remain fragile. Accordingly, this first method provides a structure which does not exhibit the various properties of a fixed plasmic membrane mentioned above.

The second method also only enables spherical liposomes to be obtained with a solid core surrounded by a lipidic bilayer which is not bonded to the solid core. This method therefore does not enable a structure to be obtained having any shape and/or possessing the properties mentioned above as well as the advantages of a fixed plasmic membrane.

The third method requires the use of a bilayer of unusual specific ionic phospholipids which alter the fundamental properties (selective impermeability and permeability and lateral fluidity) of the bilayer.

Moreover, the combination of the first and third methods described in FIG. 43 of this publication, combines the disadvantages of each of these. In all cases, these methods require the use of specific monomers and polymers and therefore do not allow an artificial membranous structure to be obtained comprising a solid substrate with various natures and shapes, which can be adapted according to the applications.

In all the text, the following terminology and definitions have been adopted:

functional membrane: any lyotropic lamellar phase, in particular a bilayer of amphiphilic compounds,

membrane fixed to a substrate: membrane bonded by ionic and/or covalent and/or lyotropic bonds to a substrate,

lyotropic bonds, complexing, and interactions: any low-energy interactions between amphiphilic compounds, organized into an ordered structure in the presence of water, in particular hydrophobic, ionic, hydrogen or Van de Waals interactions, or a combination of such interactions,

polyelectrolytic bond, complexing or interaction: bond involving, in a polar medium, a plurality of electrostatic dipoles formed of ionized groups,

artificial structure analogous to a natural fixed plasmic membrane: structure obtained by synthesis having the fundamental functional properties of a natural membrane fixed to a cytoskeleton: selective impermeability and permeability, fluid mosaic structure (lateral fluidity), polymorphism, strength, stability and an autosealable character.

In this context, the object of the invention is to provide an artificial membranous structure analogous to natural fixed plasmic membranes having a stable functional membrane (i.e. possessing two-dimensional lateral fluidity properties and selective impermeability and permeability) fixed to a substrate in the solid phase, in particular a porous substrate formed of a network of polymers, with various natures and shapes.

The object of the invention is also to provide a method for preparing such a membranous structure that is simple and compatible with industrial constraints in its implementation; a supramolecular synthetic particle or film containing such a membranous structure and applications thereof; a polymer that can serve as a bifunctional fixing compound in such a membranous structure; a method for preparing this polymer; a medicament; and therapeutic applications for this membranous structure and for these particles.

To this end, the invention concerns an artificial membranous structure analogous to fixed natural plasmic membranes, wherein it comprises:

a substrate in the solid phase having a surface provided with a surface density of electrical charges,

a stable functional membrane of amphiphilic compounds which has a free surface extending opposite the substrate, the said surface being adapted so that it can be placed in contact with a medium, a so-called external medium, with a form such that it does not circumscribe this external medium,

at least one bifunctional compound for fixing the functional membrane to the substrate, inserted between the functional membrane and the substrate, and of which the chemical structure comprises:

at least one polyionic chain adapted so as to cooperate by polyelectrolytic complexing with the surface density of electrical charges of the substrate,

at least one membranous ligand attached by a covalent bond to such a polyionic chain, and adapted to form a non-covalent stable lyotropic bond with the amphiphilic compounds of the functional membrane, without significantly affecting the functional properties of the functional membrane.

Such an artificial membranous structure thus has at the same time the properties of a functional membrane (selective impermeability and permeability and lateral fluidity) and the properties of polymorphism and mechanical properties which depend on the nature of the substrate selected.

It should be noted that according to the invention the free surface of the functional membrane extending in contact with the external medium enables exchanges with this external medium to be governed. This free surface does not circumscribe the external medium, i.e. it is not closed around this medium with which it is in contact, and this contrary to the case of a cationic liposome surrounded by a polymeric structure. In particular, the free surface of the membrane of a membranous structure according to the invention is distinct from a concave sphere, and is intended to come into contact with an external medium which is distinct from a closed internal spherical cavity. In other words, the free surface of the membrane of the membranous structure according to the invention is such that it can have a theoretical enveloping surface which extends integrally opposite the substrate and which is planar or convex with a convexity pointing in a direction away from the substrate. In general, the free surface of the membrane will itself be planar or convex with a convexity pointing away from the substrate. Nevertheless, in certain special cases, it is possible for this free surface to have concave parts with a concavity pointing outwards.

The functional membrane of the membranous structure according to the invention thus provides a protective barrier with predetermined selective impermeability and permeability which separates and protects the bifunctional fixing compounds and the substrate as well as their polyelectrolytic interactions from the external medium. Moreover, the functional membrane, and more generally the membranous structure, is perfectly stable and can be manipulated without any special precautions, including the case where the substrate is porous.

Advantageously and according to the invention, the functional membrane of a membranous structure is artificial, i.e. it is not derived from a living organism. The amphiphilic compounds of the functional membrane may be formed of any amphiphilic compounds, preferably neutral overall (not bearing a net electrical charge), which are capable of forming a functional membrane, in particular a bilayer, exhibiting the fluid mosaic state. The membrane mainly consists of amphiphilic compounds organized in a bilayer, but it may nevertheless incorporate any other compound, in varying proportions, having specific properties and which is compatible with the bilayer, i.e. preserving the functional properties and stability thereof.

Advantageously and according to the invention, the amphiphilic compounds of the membrane are phospholipidic compounds of natural or synthetic origin—in particular of the family of the phosphatidylcholines—with fatty chains comprising between 12 and 22 saturated or unsaturated carbon atoms, in particular between 16 and 18 carbon atoms.

The amphiphilic compounds of the membrane may include, other than phospholipids, a varying proportion of compounds belonging to the following families, alone or in a mixture; sphingomyelin; gangliosides; glycolipids; ceramides; di 0-alkyl; sterols (cholesterol, ergosterol etc).

The membranous structure according to the invention may include a single bifunctional fixing compound, or as a variant, a mixture of several bifunctional fixing compounds of distinct natures.

Advantageously and according to the invention, the membranous structure includes at least one bifunctional fixing compound of which the chemical structure comprises at least one plurality of membranous ligands, in particular a plurality of similar or identical membranous ligands. According to the invention, these membranous ligands are distributed over the molecule of the bifunctional fixing compound at a distance from each other which is greater than that separating the amphiphilic compounds which are contiguous to each other in a layer of the functional membrane so that this membrane has amphiphilic compounds which are not bonded to a membranous ligand.

Moreover, advantageously and according to the invention, the bifunctional fixing compound has a number of unitary ionic charges of the same sign adapted to cooperate by means of polyelectrolytic complexing with the surface density of electrical charges of the substrate, which is greater than the number of membranous ligands.

Advantageously and according to the invention, the ionic charges are distributed over the molecule of the bifunctional fixing compound at a distance from each other which is less than the smallest distance separating two membranous ligands.

Advantageously and according to the invention, the membranous ligands are selected from among phospholipids, fatty acids, isoprenoids, peptides, fatty amines, ethers, sterols, terpenes, glycolipids, shingolipids, gangliosides and ceramides.

In particular, the membranous ligands may be composed of a lipidic—in particular phospholipidic—branch similar to the amphiphilic compounds of the functional membrane, and of a bonding branch connecting this lipidic branch by means of covalent bonds to the polyionic chain of the bifunctional fixing compound. In this way, the lipidic branch of the membranous ligand is inserted in the layer facing the bilayer forming the functional membrane, and is thus bonded by lyotropic interactions within this functional membrane.

Advantageously and according to the invention, the bifunctional fixing compounds are formed of polyionic oligomers or polymers. As polycationic bifunctional fixing compounds which can be used in the invention, reference may be made to: proteins and polycationic peptides; polycationic oligo- and polysaccharides; polyamines; polycationic synthetic polymers. As polyanionic bifunctional fixing compounds which can be used in the invention, reference may be made to: polyanionic proteins and peptides; polyanionic oligo- and polysaccharides; polyacids; polyanionic synthetic polymers.

Moreover, advantageously and according to the invention, the electrical charges on the substrate are negative, and the bifunctional fixing compounds have a polycationic structure. Nevertheless, the reverse is possible.

Advantageously and according to the invention, the structure includes at least one bifunctional fixing compound of which the chemical structure includes at least one group selected from a peptide, a polypeptide, a protein or an oside. In particular, advantageously and according to the invention, all the bifunctional fixing compounds of the membranous structure according to the invention have this chemical structure, so that the membranous structure is biocompatible.

Moreover, advantageously, the membranous structure according to the invention is characterized in that it includes at least one polyamine as the bifunctional fixing compound. Indeed, such a polyamine is a relatively common compound which is easy to manipulate on a scale which includes an industrial scale.

Advantageously and according to the invention, a polyamine is used of which the chemical structure includes at least one group selected from a peptide, a polypeptide, a protein or an oside. In this way, the bifunctional fixing compound has the advantage of good biocompatibilty and can easily be manipulated on the industrial scale and at low cost. In particular, a membranous structure according to the invention advantageously includes a succinophospholipidic polylysine, i.e. of which part of the amine groups carry a succinophospholipidic ligand—in particular N-succinyl- phosphatidylethanolamine—as the bifunctional fixing compound.

Advantageously and according to the invention, the polyamine —in particular the succinophospholipidic polylysine—has a degree of grafting of the amine functional groups by the membranous ligands of between 1% and 20%. Advantageously and according to the invention, the succinophospholipidic polylysine has a molecular weight for the starting polylysine of between 10000 and 50000.

The solid substrate of a membranous structure according to the invention may be selected from among the following solids:

polyanionic substrates:

cross-linked polymers: nucleic acids, DNA, RNA; polyanionic proteins: polyaspartate, polyglutamate, sialated proteins; polyanionic polysaccharides: hyaluronic acid, alginic acid, xanthan, heparin, and acid derivatives (phosphate, sulfonate, carboxymethyl sulfate, succinate etc) of neutral polysaccharides such as cellulose, starch and dextran; synthetic polymers (nylon, silicone etc) substituted by anionic functional groups.

All these polymers can only be used in the solid and hence cross-linked form. Cross-linking may be of a covalent or ionic nature. This cross-linking must be performed before the functional membrane is established. Cross-linking may in particular be brought about by polyelectrolytic complexing between the polyanionic polymer and a polycationic polymer, it being possible for the latter to be a polycationic chain of the bifunctional compounds themselves.

portions of skin, leather, mucous membranes, superficial body growth (hair etc), cellular membrane, natural fibres (cotton, wool, paper etc)

glass, silica,

anionic tectosilicates, anionic particles and membranes for cation exchange,

polycationic substrates:

polycationic proteins: polylysine, polyarginine, protamine, histone,

polycationic polysaccharides: chitosan, DEAE dextran, synthetic polymers as their basic functional derivatives (DEAE Nylon),

alumina, cationic tectosilicates,

cationic particles and membranes for anion exchange.

Moreover, the functional membrane of a membranous structure according to the invention may have at least one compound interacting with the external medium. This interacting compound is bonded to the functional membrane by any suitable bond so as to extend within the external medium starting from the free surface of the functional membrane. Advantageously, the membranous structure includes an interacting compound selected from a peptide, a protein, a glucide and a glycoprotein. These compounds interacting with the external medium may be mono- or polyclonal antibodies, recognizing ligands (transferrin, growth factors, hormones, sugars, immunological markers), receptors, transporting proteins, enzymes or furthermore fusion proteins. An interacting compound may be bonded to at least one membranous ligand of a bifunctional fixing compound by a covalent bond, or on the other hand may be bonded by non-covalent stable lyotropic interactions with amphiphilic compounds within the functional membrane.

The functional membrane of a membranous structure according to the invention formed of a bilayer of amphiphilic compounds extends over a thickness of less than 5 nm, in particular of the order of 4 to 5 nm. Moreover, advantageously and according to the invention, the substrate has pores with an average size greater than 5 nm and less than 0.5 μm. In this way, invasion of the pores of the substrate by the bilayer of amphiphilic compounds in particular is prevented.

Such a membranous structure according to the invention may be used to obtain a medicament. Indeed, since the membranous structure according to the invention is analogous to natural plasmic membranes of the fixed type, it has their properties and can thus be used as an artificial plasmic membrane in medicaments or therapeutic compositions in particular for gene therapy.

Moreover, a membranous structure according to the invention may serve to prepare supramolecular synthetic particles. Accordingly, the invention extends to a supramolecular particle, wherein it includes a membranous structure according to the invention forming its outer periphery and delimiting an internal volume, the functional membrane of the membranous structure having a free surface that extends outside the particle, and which is intended to be placed in contact with the external medium. In a particle according to the invention, the substrate occupies at least substantially all the internal volume of the particle, or as a variant, only part of the internal volume of the particle. Moreover, the substrate may advantageously be formed of a synthetic porous polymeric matrix—in particular cross-linked DNA or RNA. The particle according to the invention may enclose a liquid composition—in particular a therapeutic composition—in its internal volume. Advantageously and according to the invention, the internal volume is entirely occupied by a substrate formed of a synthetic porous polymeric matrix which incorporates a liquid composition within its pores.

Moreover, in a particle according to the invention, the functional membrane may be adapted so as to have kinetics for the liberation of the liquid composition following a predetermined profile. It is sufficient in point of fact to select the constitution of the functional membrane in order to obtain the desired selective impermeability and permeability with a view to obtaining these kinetics, and this in a known manner (for example in the case of liposomes).

A particle according to the invention may have an average size of between 10 nm and 5 mm.

A particle according to the invention may be the subject of various applications, in particular as a medicament. Thus, the invention also extends to a medicament wherein it contains at least one particle according to the invention.

The invention also extends to a supramolecular synthetic film wherein it contains a membranous structure according to the invention. The film according to the invention may additionally incorporate an ionophore enabling ions to be selectively transported across the bilayer. Thus a film according to the invention may be formed of a portion of a membranous structure according to the invention that is not closed on itself, i.e. it is in the general form of a sheet. A film according to the invention is advantageously at least substantially planar, but may exhibit a certain amount of flexibility. A film according to the invention may be the subject of various applications, in particular for separating or extracting compounds. The invention thus also extends to the application of a film according to the invention for extracting or separating salts and/or ions from a liquid solution by filtration.

The invention also extends to a polycationic polymer with a purity above 95% formed of a polycationic polyamine provided with a plurality of lipidic—in particular phospholipidic—ligands grafted onto a part of the nitrogen atoms of the amine functional groups, and able to form a non-covalent stable lyotropic bond with a stable functional membrane of amphiphilic compounds, it being possible for this polymer to act as a bifunctional compound for fixing the functional membrane onto the substrate of a membranous structure according to the invention.

Advantageously and according to the invention, this polyamine has a degree of grafting of the amine functional groups by the membranous lipidic ligands which lies between 1% and 20%. In particular, and according to the invention the polymer is formed of polylysine, in particular a succinophospholipidic L-polylysine such as N-succinyl-phosphatidylethanolamine polylysine.

Advantageously and according to the invention, the succinophospholipidic polylysine has a molecular weight of the starting polylysine of between 10000 and 50000.

The invention also extends to the process for preparing a polycationic polymer—in particular a polymer according to the invention—provided with a plurality of lipidic ligands able to form a non-covalent stable lyotropic bond with a stable functional membrane of amphiphilic compounds, so that the polymer can act as a bifunctional compound for fixing the functional membrane onto the substrate of a membranous structure according to the invention wherein, after carrying out the chemical synthetic operations enabling the molecule of the polymer to be obtained, it is put into contact with a citrate in a polar solvent so that precipitation of the polymer is obtained.

It has indeed been surprisingly found that such a polymer may be purified in an extremely simple manner by simply adding citrate in a polar solvent in the presence of this polymer, which brings about its precipitation.

Purification of the bifunctional fixing compounds such as polylysine-NSPE's is difficult since these compounds have ionic polar parts and hydrophobic parts. As a consequence, they are not soluble either in water or in non-polar organic solvents. They are on the other hand soluble in polar organic solvents such as DMSO. These molecules interact moreover very vigorously with all the usual chromatographic supports and this much more vigorously, for example, than the starting polylysines. The usual extraction and purification techniques by liquid extraction or by chromatography are thus practically unusable.

On the other hand, precipitation with citrate has the advantage of being rapid and quantitative. The precipitate obtained is stable and may be easily washed with several types of solvent to remove contaminants. Moreover, precipitation of polycations is selective and does not entrain accompanying products.

It should be noted moreover that precipitation by citrate is easily and quantitatively reversible. Reversal is made either by adjusting the pH or the ionic strength and leads to a perfectly functional unaltered molecule. The liberated citrate is easily removed by dialysis and does not interfere with the subsequent use of the compound.

Finally, citrate is a natural cheap product which is completely non-toxic and is remarkably easy to use.

The purification method with citrate thus makes it possible to consider extracting and purifying, without any problems, bifunctional fixing compounds such as polylysine-NSPE's on an industrial scale which could not be considered with traditional methods.

The invention also extends to the method for preparing a membranous structure according to the invention wherein first of all an aqueous suspension is prepared of bifunctional fixing compounds in the following manner:

a solution of the bifunctional fixing compounds is prepared in DMSO,

an aqueous solution is prepared including at least one non-ionic detergent, at a concentration greater than its critical micellar concentration,

the solution of bifunctional fixing compounds is added to the aqueous solution.

It should be noted that the bifunctional fixing compounds such as polylysine-NSPE's are amphiphilic compounds insoluble in water and in non-polar organic solvents. When these compounds are obtained in the dry state after purification with citrate, it is practically impossible to dissolve them or even to suspend them directly in aqueous solutions even in the presence of a high concentration of non-ionic detergent. Moreover, the small amount of compound which seems to disperse does not appear to have the expected properties, namely the capacity to form polyelectrolytic complexes.

The inventors have surprisingly found that it is possible to obtain solubilization of the bifunctional fixing compounds in an aqueous medium by first of all dissolving them in DMSO and then by injecting this solution with stirring into an aqueous solution of detergent. Moreover, the bifunctional fixing compounds, such as polylysine-NSPE, solubilized in this way in DMSO, indeed possess the expected polyelectrolytic complexing properties.

In a first variant of the invention, the method is moreover characterized in that there is then added to the said aqueous suspension a composition of polyionic polymers able to form a solid substrate by polyelectrolytic cross-linking with the polyionic chains of the bifunctional fixing compounds.

Advantageously, in this first variant, the method is additionally characterized in that:

amphiphilic compounds are introduced which are able to form a functional membrane either in the aqueous solution of the detergent, or in the aqueous suspension before or after adding the composition of polyionic polymers, and so that the detergent concentration remains above its critical micellar concentration,

the detergent is then removed from the suspension.

In this first variant, the solid substrate is thus formed of a cross-linked polyionic polymer during the preparation of the membranous structure by the bifunctional fixing compounds themselves. Such is the case in particular of a substrate formed of cross-linked nucleic acid (DNA or RNA). In this variant, it is initially necessary to provide a detergent concentration greater than the CMC so as to ensure first of all the cross-linking of the substrate by formation of polyelectrolytic complexing with the polyionic chains of the bifunctional compounds, and then, secondly, during elimination of the detergent, formation of the functional membrane. It should be noted that this enables in particular the membrane to be formed outside the solid cross-linked substrate and not the reverse.

This first variant of the method for preparing the membranous structure according to the invention is more particularly applicable when the substrate is relatively small, namely of an overall mean size less than 1 μm. For example, this first variant of the preparation method according to the invention enables particles to be produced with a mean size of the order of 50 to 200 nm incorporating a DNA nucleus as the substrate, on which a functional membrane is fixed. These particles are thus artificial viruses.

The invention also extends to another variant of the method for preparing the membranous structure according to the invention which is more particularly applicable in the case where the substrate is larger, namely with an average overall size greater than 1 μm. In this second variant, the preparation method is characterized in that

amphiphilic compounds are introduced which are able to form a functional membrane either in the said aqueous solution of the detergent or in the said aqueous suspension,

the concentration of the detergent in the said aqueous suspension is then reduced to a concentration below its critical micellar concentration,

this aqueous suspension is then placed into contact with a substrate in the solid phase,

the non-ionic detergent is then removed.

It should be noted that polycationic liposomes are already known which are used for complexing polyanionic molecules such as DNA. However, since the electrostatic charges of these liposomes are situated on the outside, electrolytic complexing is propagated in the medium, which brings about a progressive aggregation of the liposomes and polyanionic molecules. This method thus produces entities with an evolutive and random structure having final properties which are not reproducible and are difficult to control.

So called “supported membranes” are also known which are formed of a lipidic bilayer on a solid planar substrate such as quartz (cf. “Supported planar membrane in studies of cell-cell recognition in the immune system” H. M. Mc Connell et al., Biochimica et Biophysica Acta 864 (1986) 95-106). In such simply supported membranes, the bilayer is not fixed to the substrate, so that the system has very great fragility, which considerably reduces the practical value.

In a variant of the supported membrane described in the above mentioned document (see also WO 89/11271), hydrophobic chains are grafted in the surface by covalent bonds on the solid support, on which a monolayer of phospholipids is deposited, and then possibly a succession of bilayers. With this system, a stable functional membrane is not formed, since the monolayer associated with the hydrophobic chains bound to the solid support cannot have the fundamental properties of a bilayer. Moreover, the lipidic bilayers which may be present above the monolayer are not fixed and are thus, here again, very fragile and unstable.

Moreover, it should also be noted that the documents “Lipophilic polylysines mediate efficient DNA transfection in mammalian cells”, Xiaohuai Zhou et al., Biochimica et Biophysica Acta 1065 (1991) 8-14 and “DNA Transfection mediated by cationic liposomes containing lipopolylysine: characterization and mechanism of action” Xiaohuai Zhou, Leaf Huang, Biochimica et Biophysica Acta 1189 (1994) 195-203, describe a lipopolylysine which is a polycationic polymer provided with lipidic chains (NGPE or DPSG), which would be obtained from a polylysine with a molecular weight of 3300.

Nevertheless, in the first of these documents, the polymer was not purified or characterized and could not be obtained in practice. Indeed, their authors wrongly consider that the total consumption of the NHS-ester of NGPE demonstrates that lipopolylysine is obtained, and do not consider purification. After all, according to these authors, the compound obtained is likely to form a clear solution in water in the absence of detergent, which is not the case, and cannot be the case, for the lipopolylysine which they describe. In practice, the structure of the product obtained in this document does not correspond to the lipopolylysine which they claim to have obtained which, from the fact that it carries two NGPE lipidic groups, would be insoluble in water and could only at best be dispersed therein.

Moreover, it should be noted that the DNA complexes and cationic liposomes described by these authors in the second document include DNA adsorbed by polyelectrolytic bonds outside the liposome complexes, (which are not in fact true liposomes) formed of lipopolylysine (LPLL) with non phospholipidic DPSG chains and dioleoylphosphatidylethanolamine (DOPE). In this structure, LPLL is thus not bonded to a functional membrane formed of a stable bilayer of amphiphilic compounds since DOPE does not form such a functional bilayer, but a hexagonal structure. Moreover, the DPSG triglyceride chains (considered as preferable to the NGPE chains which the authors rejected in this second document) could not be inserted in a functional bilayer.

The document “Drug delivery: Piercing vesicles by their adsorption onto a porous medium”, Marie-Alice GUEDEAU-BOUDEVILLE et al., Proc. Natl. Sci. USA Vol. 92, pp 9590-9592 (1995), demonstrates moreover that it is not possible to form a continuous impervious membrane with a phospholipidic bilayer on a porous substrate by direct polyelectrolytic complexing between the anionic phospholipids and a cationic support since, in this case, the phospholipidic bilayers penetrate into the pores and cover the walls thereof.

On the contrary, the invention in fact provides the only means of establishing and fixing a functional membrane continuously covering a porous substrate. The inventors have indeed found that, in a membranous structure according to the invention, the bifunctional fixing compound restricts the relative mobility of the phospholipidic bilayer with respect to the substrate by preventing its penetration inside the pores.

Other features and advantages of the invention will be apparent on reading the following description of examples and the accompanying figures in which:

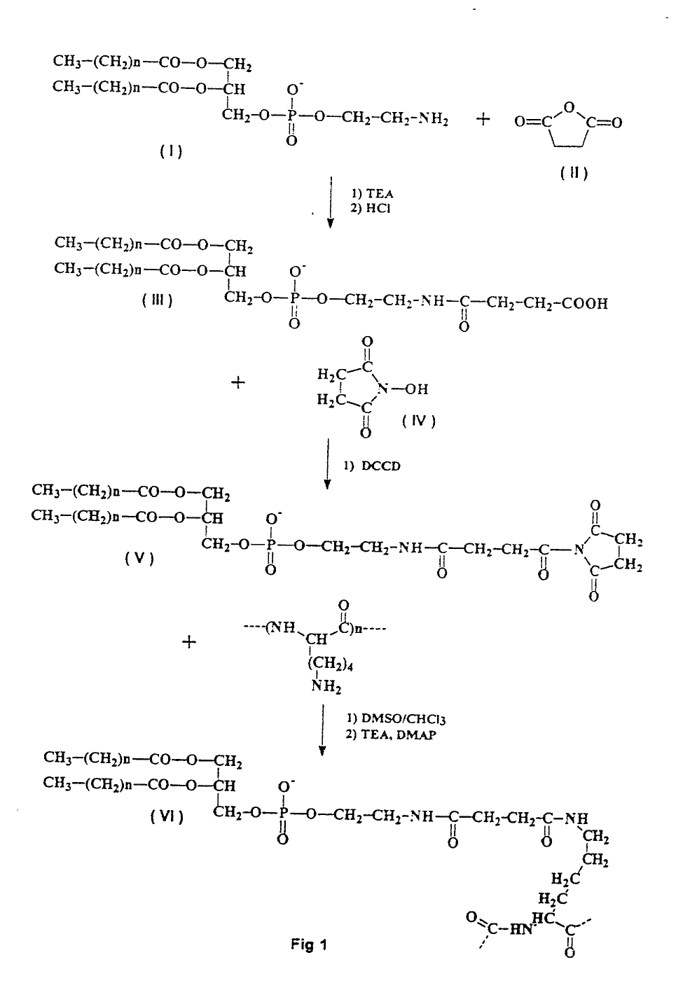

FIG. 1 is a reaction diagram of the preparation of a polycationic polymer according to the invention,

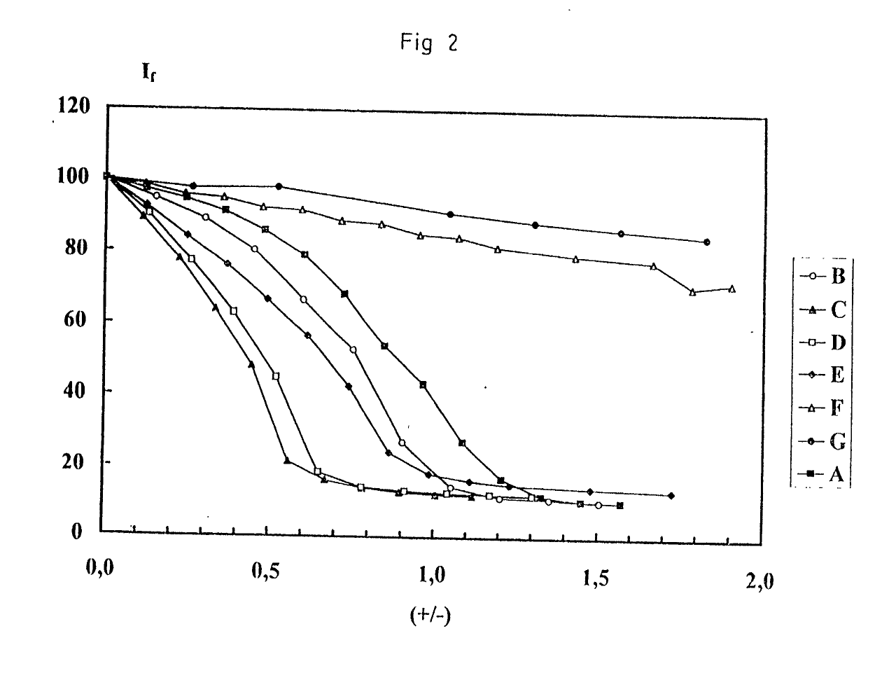

FIG. 2 is a diagram illustrating the results of the tests of example 3 on the inhibition of fluorescence by L-polylysine-NSPE dissolved in DMSO and added to an aqueous solution of DNA, detergent and BET,

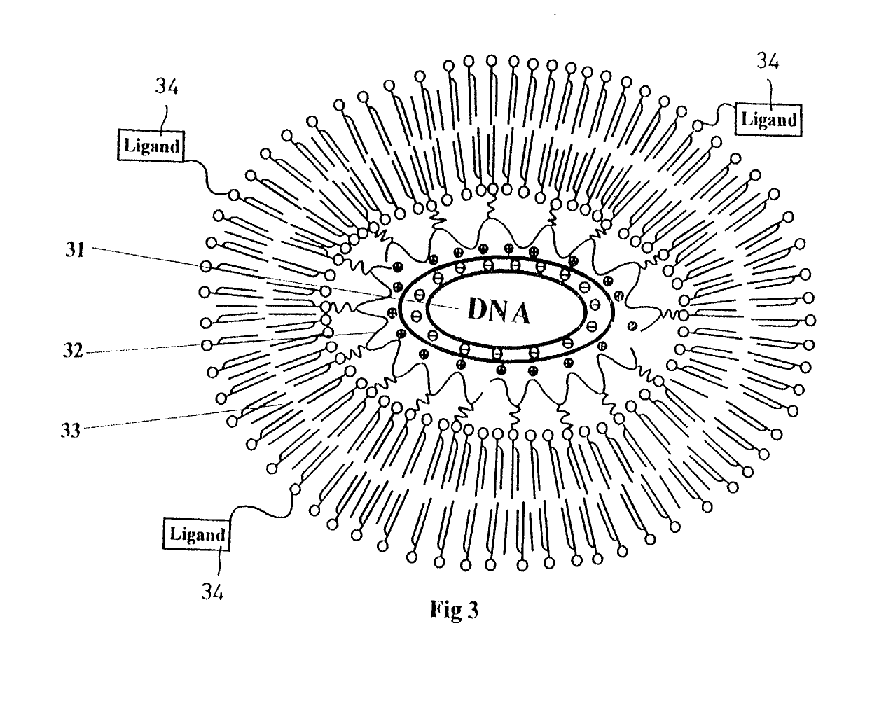

FIG. 3 is a schematic diagram illustrating the general structure of an artificial viral particle according to the invention obtained in example 4,

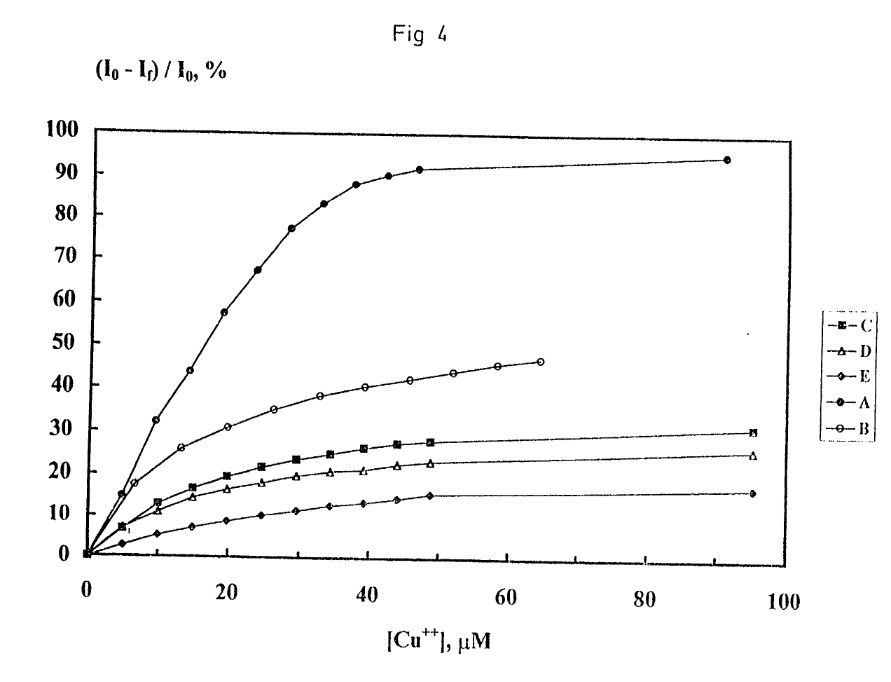

FIG. 4 is a diagram illustrating the results of tests of example 5 on the inhibition by Cu⁺⁺ of the fluorescence of artificial viral particles according to the invention,

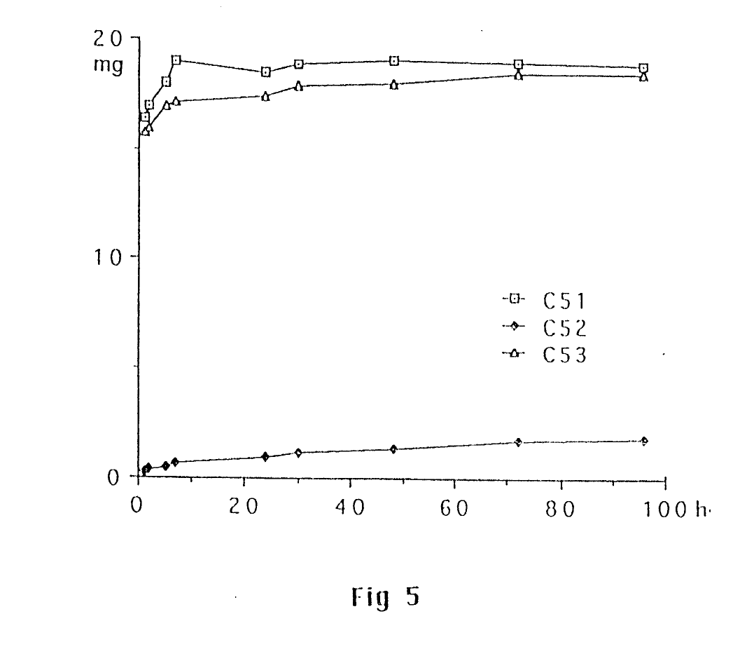

FIG. 5 is a diagram illustrating the kinetics of the liberation of haemoglobin from particles according to the invention, in accordance with example 7,

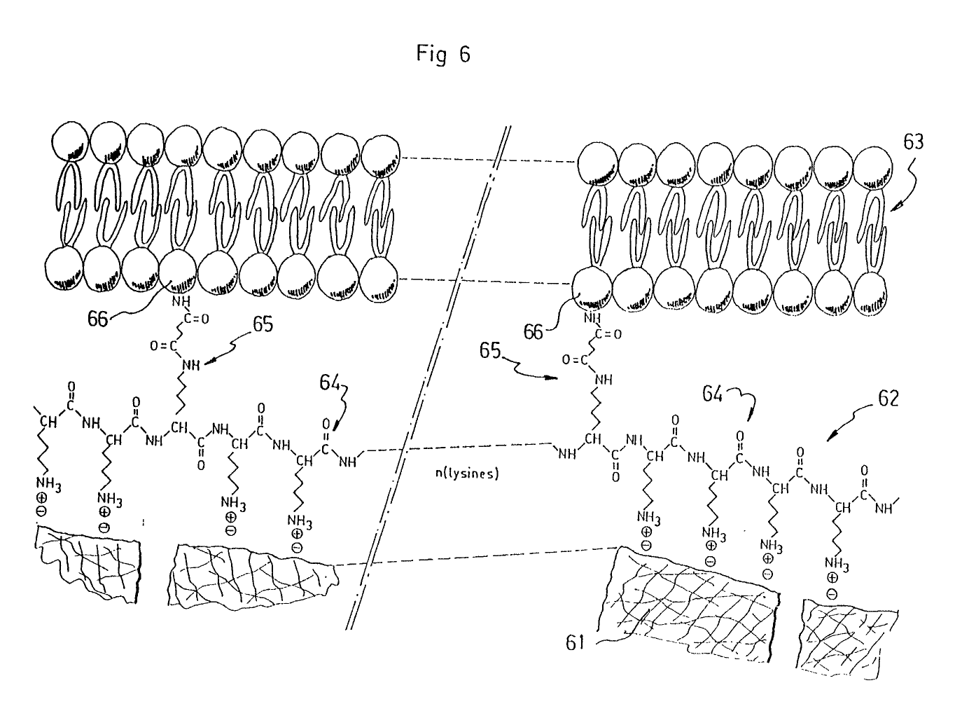

FIG. 6 is a cross-sectional partial view of a membranous structure according to one embodiment of the invention.

### EXAMPLE 1

### Preparation of a Polycationic Polymer According to the Invention: an N-succinyl-phosphatidylethanolamine L-polylysine.

1) Synthesis of N-succinyl-phosphatidylethanolamine (NSPE):

824.2 mg of egg yolk phosphatidylethanolamine EYPE (compound (I), FIG. 1) were weighed into a 25 ml flask and were dissolved in 10 ml of chloroform. 163 μl of triethylamine TEA were added with magnetic stirring. 176.7 mg of succinic anhydride (II) were then added and the reaction was allowed to continue for two hours. The disappearance of free amines was followed by chromatography on silica gel with chloroform/methanol/water mixture (1/2/0.9; v/v/v) as the eluent. 20 ml of methanol and 9 ml of water and finally 60 μl of 5N HCl were added to the reaction mixture with magnetic stirring. The pH was verified as having a value of 3 to 4. The reaction mixture was left stirring for 10 min at room temperature and 10 ml of chloroform and 10 ml of H₂O were then added and stirring was continued. The mixture was centrifuged for 10 min at 4000 rpm (418.9 rad/s) and the upper phase was aspirated off. The organic phase was removed and the aqueous phase was washed once with 10 ml of chloroform. This was centrifuged again for 10 min, the aqueous phase was removed and the organic phase was recovered. The two chloroform phases used for extraction and washing were mixed. The solvent was evaporated off in a rotary evaporator and 1 g of N-succinyl-phosphatidylethanolamine was obtained having a waxy appearance (product (III) in FIG. 1).

2) Synthesis of the Activated N-succinyl-phosphatidylethanolamine-N-succinimide Ester (NSPE-NS):

The product (III) was dissolved in 15 ml of chloroform and 560 mg of N-hydroxysuccinimide (compound (IV)) were added with magnetic stirring. 1.457 g of dried N,N′-dicyclohexylcarbodiimide (DCCD) were then weighed and dissolved in 6 ml of chloroform. 1 ml of the DCCD solution was then added progressively every 10 min at room temperature to the solution of III+IV, with magnetic stirring. After the final addition of DCCD, the mixture was allowed to incubate overnight at room temperature. The reaction mixture was filtered through glass wool to remove the dicyclohexylurea precipitate. The volume of chloroform was reduced to 3 ml by evaporation under reduced pressure and the reaction medium was once again filtered. 20 ml of acetone were added and the mixture was stirred and stored for 12 h at −20° C. The precipitate was then centrifuged off and the supernatant was recovered and evaporated. The mixture contained 800 mg of NSPE-NS (V) which was taken up in 10 ml of chloroform.

3) Synthesis of L-polylysine N-succinyl-phosphatidylethanolamine (VI).

20.4 mg of L-polylysine with a molecular weight of 19200 were weighed and dissolved in 3 ml of DMSO with magnetic stirring. 13.8 μl of triethylamine TEA and 12 mg of N-N′-dimethyl aminopyridine (DMAP) were added. 870 μl of chloroform, followed by 130 μl of solution (V), were added to the mixture. The mixture was incubated with magnetic stirring for 30 min at room temperature and then for 10 min at 50° C.

The reaction diagram of the first three steps of the synthesis are illustrated in FIG. 1. It enables the product (VI) to be synthesized, which is an L-polylysine N-succinyl-phosphatidylethanolamine, namely an L-polylysine of which certain amine groups carry NSPE phospholipidic ligands.

In all the text, such a phospholipidic polylysine is designated by polylysine-NSPE or, when it is desired to specify its molecular weight and degree of grafting by phospholipidic ligands, by the designation: L-polylysine(x)-NSPE-dsy where x is the molecular weight (in kilodaltons) of the starting L-polylysine in the form of the hydrobromide and y is the degree of grafting expressed as a percentage of substituted amine functional groups. 4) Precipitation by Sodium Citrate:

0.1 ml of a solution of 0.33M trisodium citrate, pH 7, was added to the reaction medium obtained in 3) containing 3 ml of DMSO and 1 ml of chloroform. The precipitate was left for 12 h at +4° C. The mixture was then centrifuged for 10 min at 3000 rpm (314.16 rad/s). The supernatant was removed and the precipitate was washed with 6 ml of DMSO containing Na citrate (100 μl of a 0.33M solution, pH 7). The precipitate was left for 2 hours at −20° C., and was then centrifuged at 3500 rpm (366.52 rad/s) for 10 min.

5) Taking up in DMSO:

The precipitate was taken up in 2 ml of DMSO and then 1 ml of H₂O and finally 100 μl of 1N HCl, enabling the pH to be lowered below the pK of citric acid. The solution was homogenized with magnetic stirring and the solution became clear.

6) Dialysis of DMSO and Citrate and Freeze-Drying:

Samples were dialysed, after adding 1 ml of a 64 mM solution of HECAMEG® non-ionic detergent, at a final concentration of 20 mM, overnight at −4° C. against water at pH 7, and then for 3 h against acid water (pH 3). The sample (18.5 mg) was freeze-dried. The product (VI) was obtained in this way in the freeze-dried state at a high purity, above 95%.

7) Analysis for Proteins and Phospholipids:

The freeze-dried sample was taken up in 2 ml of DMSO in which it dissolved perfectly. The sample was subjected to a traditional analysis for proteins and phospholipids. It was found that the sample obtained contained 7.55 mg of proteins and 180 μg of phosphorus which corresponded to a degree of grafting of L-polylysine of 10% amine groups, i.e. it corresponded to L-polylysine(19.2)-NSPE-ds10.

### EXAMPLE 2

### Variation of the Degree of Grafting

The number of amine groups carrying a phospholipidic ligand over the total number amine groups of the polymer (VI) constitutes the degree of grafting.

Similar products were obtained of which the degree of grafting of amine functional groups varied between 30% (L-polylysine(19.2)-NSPE-ds30) and 1% (L-polylysine(19.2)-NSPE-ds1). The synthetic method was the same as that described for L-polylysine(19.2)-NSPE-ds10, the only difference being that the volume of the solution of (V) added to the L-polylysine(19.2) varied with the desired degree of grafting.

| | | Vol. of | |
|---|---|---|---|
| Desired degree | Vol. of | chloro- | Measured |
| of grafting | (V) | form | degree of grafting |
| L-polylysine-NSPE-ds1 | 13 | 987 | L-polylysine-NSPE-ds1 |
| L-polylysine-NSPE-ds2 | 26 | 974 | L-polylysine-NSPE-ds1.7 |
| L-polylysine-NSPE-ds4 | 52 | 948 | L-polylysine-NSPE-ds3 |
| L-polylysine-NSPE-ds20 | 260 | 740 | L-polylysine-NSPE-ds21 |
| L-polylysine-NSPE-ds50 | 651 | 349 | L-polylysine-NSPE-ds30 |

The purification procedure was identical to that described in example 1, steps 4) to 6) for L-polylysine(19.2)-NSPE-ds10.

### EXAMPLE 3

### Characterization of the Properties of polylysine-NSPE.

1) Solubility

The products (VI) obtained by synthesis in the form of a powder were essentially insoluble in water, contrary to the starting L-polylysine(19.2), thus demonstrating the chemical modification of L-polylysine. These products were also insoluble in a buffer containing a non-ionic detergent such as HECAMEG® at pH 7.

Moreover, a product obtained during a synthesis carried out under the same conditions as those described previously with L-polylysine with a low molecular weight (3900) and having a degree of substitution of 6% (L-polylysine(3.9)-NSPE-ds6) was only very slightly soluble in water but also in a solution of HECAMEG® non-ionic detergent, at a concentration of 20 mM. It was also found that this product did not interact with anionic substrates such as SEPHADEX® C50, SEPHADEX® C25, SEPHADEX® SPC25, DNA, or with sodium citrate.

On the other hand, when the product L-polylysine(19.2)-NSPE-ds10was dissolved, after having been freeze-dried, in DMSO at a concentration close to 1 mg/ml, it recovered its property of being precipitated by sodium citrate. That is to say, when 0.5 mg of the product L-polylysine(19.2)-NSPE-ds10 was dissolved in 1 ml of DMSO, addition of 200 μl of a 0.19M solution of sodium citrate brought about precipitation of a complex. This complex was centrifuged and the protein content of the supernatant was determined. Only 1% of L-polylysine(19.2)-NSPE-ds10 was found in the supernatant.

Moreover, when the product L-polylysine(19.2)-NSPE-ds10, after having been freeze-dried, was dissolved in DMSO at a concentration close to 1 mg/ml and then dialysed against water so as to remove DMSO and to replace it with water, a clear homogeneous solution was obtained containing the product L-polylysine(19.2)-NSPE-dslO at a concentration corresponding to a protein concentration of 290 μg/ml. If 1 ml of this solution was added to 5 mg of SEPHADEX® C25, only 77% of L-polylysine(19.2)-NSPE-ds10 was found in the supernatant after homogenizing and decanting the SEPHADEX® C25. If 1 ml of this solution was added to 5 mg of SEPHADEX® SPC25, only 33% of L-polylysine(19.2)-NSPE-ds10 was found in the supernatant after homogenizing and decanting the SEPHADEX® C25. If 1 ml of this solution was added to 200 μl of a 0.18M solution of sodium citrate, only 38% of L-polylysine(19.2)-NSPE-dslO was found in the supernatant after homogenizing and centrifuging the complex. If 1 ml of this solution was added to 200 μl of a solution of 1 mg/ml of DNA, only 31% of L-polylysine(19.2)-NSPE-ds10 was found in the supernatant after homogenizing and centrifuging the complex. If 1 ml of this solution, with the addition of a non-ionic detergent (HECAMEG®) at a concentration of 20 mM, was added to 200 μl of a 1 mg/ml solution of DNA, only 30% of L-polylysine(19.2)-NSPE-ds10 was found in the supernatant after homogenizing and centrifuging the complex.

2) Capacity of polylysine-NSPE's to Form a Polyelectrolytic Complex with DNA:

The capacity of polylysine-NSPE's, synthesized as described previously in example 1, to interact with DNA was more precisely studied by their property of displacing a fluorescent probe, ethidium bromide (ETB), which interpenetrates naturally between the bases of DNA. In the presence of L-polylysine, ETB is displaced by the DNA/ETB complex and loses its fluorescence. This loss of fluorescence is represented by the curves of FIG. 2 as a function of the quantity of L-polylysine added, expressed in abscissae by the ratio (+/−) between the positive charges of L-polylysine and the negative charges of DNA. Curve A represents the displacement of ETB by L-polylysine(19.2) unreacted with a phospholipid, curve B represents the displacement of ETB by L-polylysine(19.2)-NSPE-ds1, curve C represents the displacement of ETB by L-polylysine(19.2)-NSPE-ds1.7, curve D represents the displacement of ETB by L-polylysine(19.2)-NSPE-ds3, curve E represents the displacement of ETB by L-polylysine(19.2)-NSPE-ds10, curve F represents the displacement of ETB by L-polylysine(19.2)-NSPE-ds21, and curve G represents the displacement of ETB by L-polylysine(19.2)-NSPE-ds30. It will thus be observed that the efficiency of the replacement of ETB is a maximum for L-polylysine(19.2)-NSPE-ds1.7 (curve C) and for L-polylysine(19.2)-NSPE-ds3 (curve D). On the other hand, if the degree of grafting is too high (greater than 20%) L-polylysine-NSPE does not associate with DNA.

### EXAMPLE 4

### Preparation of Artificial Viral Particles According to the Invention:

A lipidic solution was prepared in a 50 ml flask, in 1 ml of chloroform containing 250 μg of egg yolk lecithin(egg yolk L-α-phosphatidylcholine, EPC—Lipoid) and 25 μg of cholesterol. This solution was dried under nitrogen and was then freeze-dried for 12 h.

There was added to this dried product, 4 ml of a pH 7 buffer containing: 10 mM HEPES (N-(2-hydroxyethyl)piperazine-N′-(2-ethane sulfonic) acid) and 20 mM HECAMEG® non-ionic detergent (6-O-(N-heptylcarbamoyl)-methyl-α-D-glucopyranoside), and this mixture was subjected to ultrasound for 10 min. A clear, homogeneous solution was obtained.

While still continuing the ultrasound, 13.8 μg of L-polylysine(19.2)-NSPE-ds10 dissolved in 3 μl of DMSO (4.6 mg/l solution in DMSO) were added with the aid of a Hamilton syringe. The solution remained homogeneous and clear.

38 μg of DNA dissolved in 27 μl of water were then added with the aid of a Hamilton syringe and the mixture was left, with stirring, for 30 min. Cross-linking of the DNA with polylysine-NSPE was then produced.

The detergent was then dialysed against 5 l of distilled water with a pH of 7 for 4 h, the dialysis bath being renewed three times, which brought about the formation of the functional membrane around the DNA and the polylysine-NSPE.

Artificial viral particles were thus obtained such as shown diagrammatically in FIG. 3, comprising a DNA nucleus 31 acting as a polyanionic solid substrate, a bifunctional fixing compound 32 formed of polycationic L-polylysine-NSPE, and a peripheral external functional membrane 33 formed of a bilayer of phospholipids. The DNA nucleus 31 may be considered as an artificial nucleocapsid. The artificial viral particles were stable for at least 15 days.

### EXAMPLE 5

Demonstration of the Transfection Properties of Artificial Viral Particles. Coupling of a Cellular and Intracellular Targeting Interaction Compound, a Defective Adenovirus, to the Outer Surface of the Artificial Viral Particles.

Viral particles were synthesized in the same manner as in example 4, but adding to the phospholipid composition 5% in moles of N-{-4-(N-maleimidomethyl)cyclohexane-1-carbonyl} egg yolk phosphatidylethanolamine (MCC-EYPE). Neutravidine substituted with N-succinimidyl-3-(2-pyridyldithio)propionate (SPDP) was grafted onto the MCC-EYPE residues present on the outer surface of the particles. MCC-EYPE was obtained from EYPE (egg yolk phosphatidylethanolamine) and SMCC (succinimidyl 4-(N-maleimidomethyl)cyclohexane-1-carboxylate) as follows.

113.7 mg of EYPE were dissolved in 5 ml of anhydrous chloroform. 24 μl of triethylamine (TEA) were added and then 50 mg of SMCC dissolved in 0.5 ml of dimethylsufoxide (DMSO). The mixture was incubated for 2 hours at 40° C. with stirring. The appearance of MCC-EYPE was followed by thin layer chromatography on silica gel. The product was extracted with a chloroform/methanol/water mixture. After centrifuging for 10 min at 4000 rpm, the aqueous phase was removed and the chloroform phase containing MCC-EYPE was evaporated down. The structure of MCC-EYPE was characterized by nuclear magnetic resonance.

Preparation of N-propionyl-thiol-neutravidine (thiolated neutravidine): 10 mg of neutravidine were dissolved in 1 ml of a pH 7.9, 200 mM Hepes, 300 mM NaCl buffer. The suspension was passed through a SEPHADEX® G25 filtration gel column, at the outlet from which 500 μl fractions were collected. 95% of the protein was recovered in fractions 7 to 10. A 29 mM ethanol solution was prepared SPDP (N-succinimidyl-3-(2-pyridyldithio)propionate) and 57 μl were added to neutravidine. The mixture was incubated for one hour at room temperature and then washed on SEPHADEX® G25 with a pH 7.2, 0.1 M phosphate buffer. The neutravidine-PDP was treated with 100 μl of a 0.1 M solution of dithiothreitol for 10 min at room temperature. The thiolated neutravidine was washed on a SEPHADEX® G25 column with a 0.1 M, pH 7.2 phosphate buffer. The degree of grafting was 10 thiols per molecule of neutravidine.

Fixing of neutravidine on artificial viral particles: 3 ml portions of thiolated neutravidine were immediately incubated overnight with gentle stirring at room temperature with 1 ml of a suspension of artificial viral particles prepared with MCC-EYPE as described above. The unreacted neutravidine was removed by a filtration gel on a sepharose column.

Fixing of biotin on defective adenovirus particles: a 400 μM solution of biotin-NHS was prepared in a pH 7.9 buffer containing 5 mM Hepes, 150 mM of NaCl and 10% glycerol. 2.5×10⁹ adenovirus particles were added to 1 ml of this solution and the whole was left for 3 hours at room temperature with gentle stirring. The unreacted biotin was removed by three successive ultrafiltration passes (10 min at 1500 g). The biotin-treated adenoviruses were taken up in 1 ml of PBS buffer (Phosphate Buffer Saline, 10 mM phosphate, 150 mM NaCl, pH 7.4).

Coupling of defective biotin-treated adenoviruses with neutravidine treated artificial viral particles: a quantity of artificial viral particles corresponding to 5 μg of DNA was incubated for one hour at room temperature with gentle stirring with 8×10⁸ adenoviral particles. The suspension was adjusted to 500 μl with PBS.

Transfection with artificial viral particle-adenovirus complexes: transfections were carried out in 35 diameter containers or in multi-well plates where the wells were of the same size. Cells were transfected with 80% confluence (approximately 8×10⁵ cells per well). The 500 μl portions of the suspension obtained in the previous step were deposited in a homogeneous manner on the cells. After 1 hour's incubation, the medium was replaced by 2 ml of culture medium to which normal saline had been added. The cells were incubated for 48 hours at 37° C. in order to observe a transitory expression.

Results: no transfection was observed with particles not linked to the adenovirus. A significant level of transfection was on the other hand observed (greater than 2%) with complexes formed of artificial viral particles and adenoviruses.

This experiment showed that the artificial viral particles were quite capable of delivering DNA at the intracellular level and of enabling its expression to take place. It indicated moreover that, contrary to cationic synthetic vectors, the presence of specific ligands (recognition and fusion) at the surface of particles was indispensable for the intracellular provision and expression of the transgene.

### EXAMPLE 6

### Coupling a Targeting Interactive Compound Transferrin, to the Outer Surface of Artificial Viral Particles.

Viral particles were first of all synthesized in the same manner as in example 4 but adding 2% of dipalmitoyl phosphatidylethanolamine (DPPE) to the phospholipidic composition.

Preparation of viral particles with a membrane containing DPPE 3-mercaptopropionate. 12 mg of particles containing 10 mg of phospholipids and 200 μg of DPPE (0.26 μmol) were dispersed in 30 ml of a 75 mM sodium acetate buffer brought to pH 8.5 by adding a bicarbonate buffer. 100 μl of a 15 mM solution of succinimidyl 3-(-2-pyridyldithio) propionate (SDDP) were added and vigorous stirring was carried out for 1 h. 6 ml of a 1M sodium acetate solution were then added. The suspension was then dialysed against a 20 mM sodium acetate buffer. 2.3 mg (15 μmol) of dithiothreitol in a sodium carbonate buffer were added and the suspension was kept under argon at pH 7.5 for 1 h. The pH was then adjusted to 5.2 by adding a sodium acetate buffer and the suspension was then dialysed against a 20 mM sodium acetate buffer. A preparation was obtained containing 0.1 μmol of DPPE modified by mercaptopropionate.

Preparation of Transferrin 3-(2-pyridyldithio) Propionate.

200 μl of an ethanol solution of SPDP (3.0 μmol) were added to a solution of 120 mg (1.5 μmol) of chicken transferrin in 3 ml of a 100 mM sodium phosphate, pH 7.8. The solution was stirred vigorously for 1 h at room temperature and was then filtered through gel on SEPHADEX® G25 to give 6 ml of a solution of 1.4 μmol of transferrin modified by 2.8 μmol of dithiopyridine.

Conjugation of Transferrin with the Particles.

1 μmol of modified transferrin dissolved in a 100 mM, pH 7.8, phosphate buffer was mixed with particles containing 0.1 μmol of DPPE 3-mercaptopropionate and dispersed in a 20 mM sodium acetate buffer. The preparation was stirred for 24 hours at room temperature and was then subjected to ultrafiltration through a 100 KD membrane to remove excess transferrin.

Viral particles were obtained such as those represented in FIG. 3, provided with ligands 34 formed of transferrin.

### Example 7

### Characterization of Artificial Viral Particles

1) Synthesis of fluorescent L-polylysine: L-polylysine(19.2)-fluorescein-ds0.4.

36.3 mg of L-polylysine with a molecular weight of 19200, were weighed into 25 ml flask and were dissolved in 10 ml of DMSO with magnetic stirring. 40 μl of triethylamine were added and the solution was left for 10 min. 1.1 mg of fluorescein isothiocyanate (FITC) were then added, dissolved in 149 μl of dimethylformamide (DMF). The reaction continued at 30° C. for 2 h. The product was analyzed by chromatography on silica gel which showed the disappearance of free FITC and the appearance of a fluorescent protein product in the deposit. The L-polylysine-fluorescein was purified as follows. The DMSO of the reaction medium was dialysed twice for 2 h against distilled water at pH 6.5. The dialysed product was then incubated with 500 mg of SEPHADEX® C50 in 100 ml of distilled water and then deposited on a column. The column was first of all washed with 100 ml of distilled water at pH 7. The L-polylysine-fluorescein was eluted in the column with 100 ml of a 2M NaCl solution at pH 9. This solution was dialysed against distilled water. The final solution contained 35 mg of protein. The quantity of fluorescein was estimated by spectrometry at 496 nm with a molecular extinction coefficient of 90000 m⁻¹cm⁻¹. The degree of grafting was 1/233 of amine functional groups.

2) Characterization of Viral Particles by Fluorescence Inhibition:

The capacity of polylysine-NSPE's synthesised as described previously to enable an impermeable membranous structure to be established with cupric (Cu⁺⁺) ions surrounding a DNA complex, was studied by the method of inhibiting the fluorescence of a fluorescent probe attached to the complexed DNA. This probe was the L-polylysine(19.2)-fluorescein-ds0.4 obtained in 1). The viral particles were prepared as described in example 4 with the sole difference that L-polylysine-NSPE-ds0.1 was replaced by a mixture of 90% L-polylysine(19.2)-NSPE-ds(n) and 10% L-polylysine(19.2)-fluorescein-ds0.4. The mixture (phospholipids+cholesterol+detergent+L-polylysine-NSPE-ds(n)+L-polylysine(19.2)-fluorescein-ds0.4+DNA) was then dialysed and the fluorescence of the particles was analyzed with a spectrofluorimeter. The intensity of the fluorescence was analyzed during the progressive addition of Cu⁺⁺. The values shown as ordinates on FIG. 4 show the degree of inhibition of fluorescence, expressed as a percentage, and calculated as follows: (I₀-I_{f})/I₀ where I₀ is the intensity of fluorescence in the absence of copper and I_{f} is the intensity of fluorescence in the presence of copper.

Curve A shows the degree of inhibition of fluorescence of particles obtained from L-polylysine-NSPE-ds10, but in the presence of detergent, which impedes the establishment of a functional membrane around the particle. It will be observed that the degree of inhibition of the fluorescence of L-polylysine(19.2)-fluorescein-ds0.4 reached a value close to 100% for a Cu⁺⁺ concentration of 50 μM. Curve B shows the degree of inhibition of the fluorescence of particles obtained from L-polylysine-NSPE-ds1 after dialysis of the detergent. It will be observed that the degree of inhibition of the fluorescence of L-polylysine(19.2)-fluorescein-ds0.4 reached a value close to 30% for a Cu⁺⁺ concentration of 50 μM. Curve D shows the degree of inhibition of fluorescence of particles obtained from L-polylysine-NSPE-ds10 after dialysis of the detergent. It will be observed that the degree of inhibition of the fluorescence of L-polylysine(19.2)-fluorescein-ds0.4 reached a value close to 25% for a Cu⁺⁺ concentration of 50 μM. Curve E shows the degree of inhibition of fluorescence of particles obtained from L-polylysine-NSPE-ds21 after dialysis of the detergent. It will be observed that the degree of inhibition of the fluorescence of L-polylysine(19.2)-fluorescein-ds0.4 reached a value close to 20% for a Cu⁺⁺ concentration of 50 μM.

These results show that when the degree of substitution of L-polylysine-NSPE-ds(n)'s increases, protection of the DNA/L-polylysine-NSPE-ds(n) complex by free phospholipids increase, which demonstrates the establishment of a fixed functional membrane around the viral particle.

3) Size of Artificial Viral Particles:

Analysis of the size of artificial viral particles carried micro out with a particle/analyzer, demonstrated the presence of particles having a mean size of 100 nm.

4) Application of Artificial Viral Particles.

Artificial viral particles may act therapeutically to provide in vivo intracellular delivery of therapeutic genes, for example for the treatment of genetic diseases (cystic fibrosis etc) or of certain cancers or for the preparation of gene vaccines.

The results obtained clearly indicate that a membrane impervious to ions was established around the DNA/polylysine complexes. They thus demonstrate the possibility of establishing a functional membrane according to the invention. They also indicate that polyelectrolytic complexes are indeed isolated from the external medium and no longer have the possibility of propagating in the external medium.

### EXAMPLE 8

### Preparation of Supramolecular Synthetic Particles Containing Haemoglobin absorbed in a Porous Substrate (Artificial Erythrocytes).

In this example, supramolecular synthetic particles were prepared from a polycationic polylysine-NSPE polymer.

a) Association of L-polylysine-NSPE and Phospholipids Forming a Bilayer:

9 mg of EYPC phospholipids (egg yolk phosphatidylcholine) and 1 mg of cholesterol were dissolved in 1 ml of chloroform which was evaporated off under reduced pressure in a rotary evaporator. The lipids were then dispersed in 10 ml of a 40 mM aqueous solution of non-ionic HECAMEG® detergent. When dispersion was complete, 100 μl of a 40 mM non-ionic detergent solution were added containing 1 mg of L-polylysine(19.2)-NSPE-ds6.25 obtained as indicated in examples 1 and 2. The micellar preparation was treated for 1 min in an ultrasonic bath, and then dialysed against distilled water to give a suspension of polylysine-NSPE and phospholipids forming a bilayer. The presence of particles having an average size of 300 to 600 nm was observed in a particle analyzer.

b) Preparation of Neutral Reference Liposomes:

The procedure was as indicated in a), but without the addition of the polylysine-NSPE solution to the dispersion of lipids in the detergent. The presence of particles was observed having an average size of 200 to 400 nm.

c) Absorption of Haemoglobin on the Porous Substrate:

5 mg of porous particles (SEPHADEX® SPC50) with a diameter of 150 μm substituted with sulfopropyl groups and having a pore size sufficient to allow the penetration of molecules with a maximum molecular weight of 250000, were dispersed in 5 ml of a 10 mM bi-tri buffer, of pH 6.5 (where the haemoglobin is cationic and is attracted inside the anionic sites of the porous particles). 20 mg of haemoglobin, extracted from human erythrocytes by the lysis method in a hypotonic medium, were then added to this dispersion. The preparation was stirred for 24 h at 4° C. on a planetary stirrer. The particles were then decanted and the quantity present in the supernatant was determined by UV spectrometry at 410 nm. The results obtained indicated that more than 98% of the haemoglobin was present inside the particles. The non-absorbed haemoglobin was removed by decanting the particles and washing them with a 10 mM bi-tri buffer.

d) Fixing of the Functional Membrane:

The particles charged with haemoglobin (24 mg) obtained in c) were dispersed in 4 ml of a 10 mM bi-tri buffer pH 6.5 buffer. 1 ml of the suspension of polylysine-NSPE/phospholipids obtained in a) was added and stirring was continued for 2 h with a planetary stirrer at 4° C. 625 μl of a 40 mM HECAMEG® non-ionic detergent solution were then added to reach a final concentration of 5 mM. The suspension was stirred again for 5 min. and the particles were then decanted off and the cake washed with distilled water to remove the detergent and excess polylysine-NSPE/phospholipid complexes.

e) A Control Experiment was Carried out as Indicated in d) but Using the Neutral Reference Liposomes Obtained in b) in place of the Suspension Obtained in a).

The results obtained demonstrated that the membranous structure of the particles according to the invention, as in the case of natural erythrocytes, was the only barrier preventing haemoglobin leaving the particle.

### EXAMPLE 9

### Study of the Liberation of Haemoglobin

In this example, the liberation was studied, in a comparative manner, of haemoglobin absorbed inside the porous particles obtained in example 8.

The particles charged with haemoglobin were incubated in 5 ml of a 150 mM PBS, pH 7.4, buffer (where the haemoglobin was above its isoelectric point and therefore had the tendency to be excluded from the porous substrate). Stirring was carried out at 4° C. with a planetary stirrer. Aliquots were withdrawn at regular intervals of time and the concentration of haemoglobin present in the supernatant was determined by spectrophotometry.

FIG. 5 illustrates the curves for the kinetics of the liberation of haemoglobin obtained with time, with the ordinates as the mass in mg of haemoglobin liberated and with the abscissae as the time in hours. Curve C51 corresponds to the result obtained with the particles simply charged with haemoglobin resulting from step c) of example 7. Curve C53 corresponds to the results obtained with the particles obtained in step e) of example 8 (neutral liposomes obtained in b) put into contact in the suspension of polylysine-NSPE/phospholipids obtained in a)). Curve C52 corresponds to the results obtained with the particles obtained in d) of example 8. As will be seen, in the absence of fixed functional membranes (curves C51 and C53) haemoglobin was liberated quantitatively from the particles from the start of incubation. On the other hand, with particles according to the invention (curve C52) provided with a fixed functional membrane, haemoglobin was not liberated and remained associated inside the porous particles. These results thus demonstrate that the fixed functional membrane enables haemoglobin to be retained efficiently.

### EXAMPLE 10

### Preparation of Supramolecular Synthetic Particles Containing an Anti-Cancer Drug

The overall procedure was as in example 7, but incorporating doxorubicin (C27H29NO11,PM543, 53) in place of haemoglobin, 5 mg of doxorubicin hydrochloride being dissolved in 1 ml of distilled water and incubated with 5 mg of porous particles (SEPHADEX® C25) substituted with carboxymethyl groups. The pore size of the particles was such that molecules with a maximum molecular weight of 25000 could penetrate. The suspension was stirred for 2 h at 4° C. with a planetary stirrer. It was then decanted and the free doxorubicin concentration was measured by UV spectrophotometry at 480 nm. The results obtained indicated that more than 97% of the doxorubicin was associated with the particles. The non-absorbed doxorubicin fraction was removed by decanting and washing the cake with distilled water.

The particles charged with doxorubicin were dispersed in 4 ml of distilled water. 1 ml was then added of a polylysine-NSPE suspension and phospholipids forming the bilayer obtained in step a) of example 7, and gentle stirring was carried out for 2 h at 4° C. 625 μl of a 40 mM HECAMEG® non-ionic detergent solution were then added, stirring was carried out for 5 min and the particles were decanted off and the cake was washed with distilled water to remove the detergent and excess polylysine-NSPE/phospholipid complexes.

As in example 8, a control experiment was carried out using a suspension of neutral liposomes composed solely of phospholipids (EYPC) and cholesterol.

### EXAMPLE 11

### Study of the Liberation of Doxorubicin

The kinetics were studied of the liberation of doxorubicin by the particles obtained in example 10.

The particles were dispersed in 150 ml of a 150 mM PPS buffer, of pH 7.1, and gently stirred. Aliquots were withdrawn at regular intervals of time and the concentration of doxorubicin present in the supernatant was measured by/spectrophotometry at 480 nm.

The results obtained indicated that, at the end of 1 h, 45% by weight of the doxorubicin was liberated from the particles of the control experiment and from the particles charged with doxorubicin used before contact with the suspension of polylysine-NSPE/phospholipids, i.e. particles lacking a fixed functional membrane. On the other hand, with the particles according to the invention having a fixed functional membrane, only 5% of the doxorubicin was liberated. Thus the fixed functional membrane formed of phospholipids enabled the doxorubicin to be retained efficiently inside the particles according to the invention.

### EXAMPLE 12

### Preparation of Small-Sized Supramolecular Synthetic Particles

The procedure was as in example 10, but an attempt was made to prepare smaller sized porous particles. 1 g of SEPHADEX® C25 porous matrix was dispersed in 100 ml of distilled water and the preparation was ground for 15 min with a helical mill. The preparation was then centrifuged at 3000 g for 10 min and the supernatant was recovered and then subjected to further centrifuging at 25000 g for 45 min. The supernatant was removed, the cake was put back into suspension and 50 mg of a dry product were obtained following freeze-drying. The size of the particles obtained, measured by a particle analyzer, was between 300 and 500 nm.

5 mg of these small-size porous particles were dispersed in 5 ml of distilled water. 5 mg of doxorubicin were added and the whole was stirred for 2 h. The concentration of doxorubicin not associated with the particles was determined by ultrafiltration of an aliquot of the suspension through an ultrafiltration membrane of which the separation threshold was 50000, and the concentration of free doxorubicin was measured by UV spectrometry at 480 nm. The results obtained indicated that more than 98% of the doxorubicin was associated with the particles. These were then used as they were without supplementary purification.

In order to associate these small-size porous particles with polylysine-NSPE/phospholipid complexes capable of forming a fixed functional membrane on these particles, small-size complexes were formed. To this end, 9 mg of EYPC phospholipids and 1 mg of cholesterol were dissolved in 1 ml of chloroform which was evaporated off under reduced pressure in a rotating evaporator. The lipids were then dispersed in 2.5 ml of a 40 mM aqueous solution of non-ionic HECAMEG® detergent. When dispersion was complete, 100 μl of a solution of L-polylysine(19.2)-NSPE-ds6.25 were added, obtained as indicated in examples 1 and 2. The micellar preparation was treated for 1 min in an ultrasonic bath and was then rapidly diluted with 10 ml of distilled water and dialysed against distilled water to give a preparation of small-size polylysine-NSPE/phospholipid complexes. The presence of particles having a mean size of 50 nm was observed by a particle analyzer.

Small-size porous particles according to the invention were then prepared. 625 μl of a 40 mM aqueous solution of the non-ionic HECAMEG® detergent was added to 2.5 μl of the suspension of polylysine-NSPE/polyphospholipids prepared previously, so as to obtain a detergent concentration of 10 mM. The suspension was treated for 1 min in an ultrasonic bath and was added slowly to 2.5 ml of the small-size porous particles charged with doxorubicin prepared previously, and was then placed in a continuous dialysis cell, kept continuously stirred.

### EXAMPLE 13

### Study of the Liberation of Doxorubicin

The particles prepared in example 12 were dispersed in 150 ml of a 150 mM pH 7.4 PBS buffer and gently stirred. Aliquots were withdrawn at regular time intervals and the concentration of doxorubicin liberated was measured by UV spectrophotometry at 480 nm after ultrafiltration through a membrane having a filtration threshold of 50000.

The results obtained demonstrated that at the end of 4 h, 65% of the doxorubicin had been liberated from particles without a fixed membrane (before contact with the suspension of polylysine-NSPE/phospholipids) whereas only 10% of the doxorubicin had been liberated from particles provided with a fixed functional membrane according to the invention.

### EXAMPLE 14

### Study of the Polyelectrolytic Complexing of Polylysine-NSPE on the Porous Particles of Example 8.

Methodology

A fluorescent marker was used (marketed by the Molecular Probes Company) consisting of a phospholipid, dipalmitoylphosphatidylethanolamine, substituted by a fluorescent group, rhodamine. This compound, with a phospholipidic nature, enabled phospholipids to be revealed, either in the form of patterns when associated together, or in the form of diffuse fluorescence when they were in a soluble form or highly dispersed. Observations were made both by phase contrast and by fluorescence microscopy (λ ex 510-560 nm, λ em 590 nm).

Preparation of Reference Fluorescent Liposomes

9 mg of EYPC, 1 mg of cholesterol and 0.05 mg of dipalmitoyl phosphatidyl ethanolamine rhodamine (DPPERd) were dissolved in 1 ml of chloroform which was then evaporated off under reduced pressure. The residue was taken up in 10 ml of a 40 mM HECAMEG® solution. When the solution had become completely clear, it was dialysed against distilled water. The fluorescent liposomes obtained were very difficult to distinguish by observation and, on account of their small size, appeared in the form of a uniform background noise.

Preparation of Fluorescent Polylysine-NSPE/Phospholipid Complexes:

The procedure was as previously with addition to the solution of lipids in detergent of 100 μl of a 40 mM HECAMEG® solution containing 1 mg of L-polylysine(19.2)-NSPE-ds6. The preparation was treated for 1 min in an ultrasonic bath and then dialysed against distilled water. The fluorescent polylysine-NSPE/phospholipid complexes were larger than the reference liposomes and were visible in the form of small fluorescent spots.

Interaction between the Polylysine-NSPE/Phospholipid Complexes and Porous SEPHADEX® SPC50 Porous Particles.

The SEPHADEX® particles were first of all revealed by phase contrast microscopy. They were in the form of regular spheres with a diameter of between 100 and 150 μm. These particles (0.1 mg in 0.5 ml) were then incubated with 20 μl of the preparation of polylysine-NSPE/phospholipid complexes.

Adhesion of the first fluorescent entities to the surface of the particle could be clearly observed. At a more advanced stage of association, a dense but discontinuous corona was distinguished, but with juxtaposition of small fluorescent dots. These results indicated that, by virtue of their positive charge, the polylysine-NSPE/phospholipid complexes were attracted by the surface of the particles which was negatively charged. The fact of having a corona with a discontinuous structure moreover indicated that the entities had not fused together.

The particles obtained were then incubated with 2 ml of a 150 mM PBS NaCl buffer for 10 min. It was found by observation that the appearance of the particles remained unchanged. The energy involved in the formation of polyelectrolytic complexing is indeed considerable and this complexing remains stable over a wide range of pH and ionic strength.

The preparation was then decanted and incubated with 2 ml of a 5 mM HECAMEG® solution for 10 min. It was found that the appearance of the corona had changed and had become completely regular. This result indicated that the entities which were attached individually to the surface of the particles had now fused together to form a continuous phospholipidic bilayer fixed to the particulate substrate.

The preparation was then once more decanted and incubated with 2 ml of a 40 mM HECAMEG® solution for 10 min. It was found that the appearance of the particles had once again completely changed. It was no longer possible to reveal the particles by fluorescence. On the other hand, the particles seemed rather to appear as black on a diffusely fluorescent background resulting from the solubilization of the phospholipids by the 40 mM HECAMEG® solution. This experiment indicated that the phospholipids were indeed attached to the particles by hydrophobic interactions.

A control was in addition carried out with neutral fluorescent liposomes incubated with particles of SEPHADEX® SPC50. No interaction was found between the particles which appeared black and the liposomes which appeared as a uniform fluorescent background. This result indicated that the adhesive properties of the polylysine-NSPE/phospholipid complexes were indeed due to the presence of phospholipid-treated polylysine-NSPE.

Efficiency of Polyelectrolytic Complexing

This efficiency was illustrated by the following two experiments

1) Preparation of a Polyelectrolytic Complex between Particles of SEPHADEX® SPC25 and L-polylysine(19.2)-fluorescein-ds0.4

0,1 mg of particles of SEPHADEX® SPC25 (approximately 80 μm in size), the surface of which was covered with negative charges, were incubated with 200 μl of an L-polylysine(19.2)-fluorescein-ds0.4 solution as prepared in example 5. These particles were first of all observed by phase contrast.

Observation by fluorescence (λ ex 458-490 nm, λ em 515-565 nm) after incubation for a period of 10 s already revealed the presence of fluorescence around the particle.

In another preparation, the particles were incubated in the same manner for 5 min with the fluorescent polylysine solution. They were then washed with distilled water, incubated with 2 ml of 150 mM PBS NaCl buffer and finally washed twice with 2 ml of PBS buffer. The results of the observation indicated that the fluorescence was apparently maintained quantitatively around the particle. Similarly, no change was brought about by incubation with a 40 mM HECAMEGE solution.

2) Preparation of a Polyelectrolytic Complex between Particles of Porous Silica and Fluorescent L-polylysine(19,2).

0.1 mg of silica particles (pore size 60 Angström, particle size 40 μm) were incubated with 200 μl of the fluorescent polylysine solution for 5 min and then decanted and washed with 2 ml of distilled water. The results were observed by phase contrast and fluorescence. They indicated that the polylysine had a strong affinity for silica. Incubation of the particles with 2 ml of 150 mM PBS NaCl buffer indicated that this affinity was not affected by this buffer, and the stability of the polyelectrolytic interactions between polylysine and silica was thus also very high with a material such as silica.

These results thus indicated that silica-based materials are capable of interacting strongly with polycations such as polylysine. Accordingly, in particular, any glass-based material can be covered with a functional membrane according to the invention and can be used as a solid substrate.

### EXAMPLE 15

### Preparation of a Supramolecular Synthetic Film According to the Invention.

In this example, a phospholipidic functional membrane was fixed to a planar porous substrate formed from an ion exchange filter.

A polylysine-NSPE/phospholipid suspension was prepared as indicated in step a) of example 6, but with 20% by weight of cholesterol based on the EYPC phospholipids. 2 ml of the suspension were dissolved in 200 ml of distilled water and the mixture was placed in a 47 mm diameter ultrafiltration cell fitted with a Gelman® anionic filter (reference 60943) with a porosity of 0.445 μm and a diameter equal to 47 mm. This anionic filter thus formed the solid porous substrate of the film. The pressure of the cell was adjusted to obtain an initial flow rate of 2 ml/min. When the flow rate fell below 0.3 ml/min, indicating that the polylysine-NSPE/phospholipid complexes had covered the surface of the filter and blocked the pores, ultrafiltration was stopped. The greater part of the supernatant was removed and 100 ml of a 5 mM solution of a non-ionic detergent were added. The pressure was readjusted to obtain a flow rate of 1 ml/min until two thirds of the solution had been filtered. Addition of the 5 mM non-ionic detergent solution had the effect of forming a bilayer of phospholipids on the anionic filter. In point of fact, this non-ionic detergent introduced below its critical micellar concentration enabled the polylysine-NSPE/phospholipid complexes to be fused to the surface of the filter. The greater part of the supernatant was then removed and the filter was cautiously washed several times with distilled water while re-applying pressure so as to cause the distilled water to pass through the filter.

A control experiment was carried out using neutral liposomes as in example 8.

In addition, another experiment was prepared by fixing a functional membrane containing an ionophore (an agent facilitating the diffusion of ions) to the filter. To this end, a suspension of polylysine-NSPE/phospholipids was prepared as indicated previously in this example, but adding 0.1 mg of monensin (ionophore) to the chloroform solution of EYPC phospholipids (1% by weight of the total). The suspension was then used to establish the membrane on the filter in the same manner as previously indicated.

### EXAMPLE 16

### Study of the Impermeability of the Films to Ions.

200 ml of a 25 g/l NaCl solution, having a resistivity of 55 mS/cm, were introduced into the ultrafiltration cell. Pressure was established to obtain a flow rate of 0.5 ml/min and 1 ml fractions were collected of which the conductivity was measured. The experiments were carried out under different conditions with: a filter alone, a filter resulting from the control experiment associated with neutral liposomes, a synthetic film according to the invention formed of the filter and the fixed functional membrane and a film according to the invention formed of the filter provided with a fixed functional membrane containing an ionophore. The results obtained are expressed in the following table:

Conductivity
(as % of the conductivity of the initial NaCl solution)

| | Fractions | | | | | | | |
|---|---|---|---|---|---|---|---|---|
| | 1 | 2 | 3 | 4 | 5 | 6 | 7 | 8 |
| Nature of the film | | | | | | | | |
| Filter alone | 50 | 100 | 100 | 100 | 100 | 100 | 100 | 100 |
| Filter + | 55 | 100 | 100 | 100 | 100 | 100 | 100 | 100 |
| neutral | | | | | | | | |
| liposomes | | | | | | | | |
| Filter + fixed | 1 | 1 | 1 | 2 | 2 | 2 | 2 | 2 |
| membrane | | | | | | | | |
| Filter + fixed | 4 | 6 | 6 | 7 | 7 | 7 | 7 | 7 |
| membrane + | | | | | | | | |
| ionophore | | | | | | | | |

These results indicate that the fixed membrane indeed enabled the ions to be retained whereas the filter alone and the filter associated with neutral liposomes were completely ineffective. The results also show that this effect was partially reversed by the presence within the membrane of an ionophore compound, the function of which was to transport ions through the bilayers.

Accordingly, the fixed functional membranes according to the invention on planar supports retained the same selective permeability properties as the plasmic membranes of eucaryote cells and could, like them, extend over considerable areas while remaining functional.

A film according to the invention may be used to extract or separate salts and/or ions from a liquid solution by filtration.

FIG. 6 illustrates in detail but in a schematic manner, the composition of a membranous structure according to the invention and hence a portion of a film according to the invention. This structure comprises a bilayer 63 forming a functional membrane, polycationic L-polylysine NSPE's 62 and the porous substrate 61 formed of the filter. The L-polylysine-NSPE's form polycationic polymeric chains 64 and carry membranous ligands 65 of which the phospholipidic chains 66 are inserted by lyotropic interactions inside the functional membrane 63.

The invention may be the subject of many variants and applications. In particular, other polycationic or polyanionic polymers may be used as bifunctional compounds; other amphiphilic compounds may be used to form the membrane; and other polyionic solid substrates may be used as long as they have a surface density of positive and/or negative electrical charges.

1. An artificial membranous structure analogous to fixed natural plasmic membranes, wherein it comprises:
a substrate (31, 61) in the solid phase having a surface provided with a surface density of electrical charges,
a stable functional membrane (33, 63) of amphiphilic compounds which has a free surface extending opposite the substrate, the said free surface being adapted so that it can be placed in contact with a medium, a so-called external medium, with a form such that it does not circumscribe this external medium,
at least one bifunctional compound (32, 62) for fixing the functional membrane (33, 63) to the substrate (31, 61), inserted between the membrane and the substrate, and of which the chemical structure comprises:
at least one polyionic chain (64) adapted so as to cooperate by polyelectrolytic complexing with the surface density of electrical charges of the substrate (31, 61),
at least one membranous ligand (65, 66) bonded by a covalent bond to such a polyionic chain, and adapted to form a non-covalent stable lyotropic bond with the amphiphilic compounds of the functional membrane (33, 63), without significantly affecting the functional properties of the functional membrane (33, 63).

2. The membranous structure as claimed in claim 1, wherein the amphiphilic compounds of the functional membrane (3, 63) are phospholipidic compounds.

3. The membranous structure as claimed in one of claims 1 and 2, wherein it includes at least one bifunctional fixing compound (32, 62) of which the chemical structure comprises at least one plurality of membranous ligands (65, 66).

4. The membranous structure as claimed in claim 3, wherein membranous ligands (65, 66) are distributed over the molecule of the bifunctional fixing compound (32, 62) at a distance from each other that is greater than that separating the amphiphilic compounds which are contiguous to each other in a layer of the functional membrane (33, 63), so that the functional membrane (33, 63) has amphiphilic compounds which are not bonded to a membranous ligand (65, 66).

5. The membranous structure as claimed in either of claims 3 or 4, wherein the bifunctional fixing compound (32, 62) has a number of unitary ionic charges of the same sign adapted to cooperate by polyelectrolytic complexing with the surface density of electrical charges of the substrate, greater than the number of membranous ligands (65, 66).

6. The membranous structure as claimed in claim 5, wherein the ionic charges are distributed over the molecule of the bifunctional fixing compound (32, 62) at a distance from each other that is less than the smallest distance separating two membranous ligands (65, 66).

7. The membranous structure as claimed in one of claims 1 to 6, wherein the membranous ligands (65, 66) are selected from phospholipids, fatty acids, isoprenoids and peptides.

8. The membranous structure as claimed in one of claims 1 to 7, wherein the bifunctional fixing compounds (32, 62) are formed of oligomers or polymers.

9. The membranous structure as claimed in one of claims 1 to 8, wherein the electrical charges of the substrate (31, 61) are negative, and in that the bifunctional fixing compounds (32, 62) have a polycationic structure.

10. The membranous structure as claimed in one of claims 1 to 9, wherein it includes at least one bifunctional fixing compound (32, 62) of which the chemical structure includes at least one group selected from a peptide, a polypeptide, a protein or an oside.

11. The membranous structure as claimed in one of claims 1 to 10, wherein it includes at least one polyamine as a bifunctional fixing compound.

12. The membranous structure as claimed in claim 11, wherein the polyamine is a succinophospholipidic polylysine.

13. The membranous structure as claimed in one of claims 1 to 12, wherein the functional membrane (33, 63) has at least one compound (34) interacting with the external medium.

14. The membranous structure as claimed in claim 13, wherein it includes an interacting compound (34) selected from a peptide, a protein, a glucide and a glycoprotein.

15. The membranous structure as claimed in one of claims 13 and 14, wherein an interacting compound is bonded to at least one membranous ligand (65, 66) of a bifunctional fixing compound (32, 62) by a covalent bond.

16. The membranous structure as claimed in one of claims 13 to 15, wherein an interacting compound (34) is bonded by a non-covalent stable lyotropic bond with the amphiphilic compounds of the functional membrane (33, 63).

17. The membranous structure as claimed in one of claims 1 to 16, wherein the functional membrane (33, 63) extends over a thickness less than 5 nm.

18. The membranous structure as claimed in one of claims 1 to 17, wherein the substrate (31, 61) has pores with a mean size greater than 5 nm and less than 0.5 μm.

19. A use of a membranous structure as claimed in one of claims 1 to 18 for obtaining a medicament.

20. A supramolecular synthetic particle wherein it comprises a membranous structure as claimed in one of claims 1 to 18 forming its outer periphery and delimiting an inner volume, the functional membrane (33) of the structure having a free surface which extends outside the particle and which is intended to be placed in contact with an external medium.

21. The particle as claimed in claim 20, wherein the substrate (31) occupies at least substantially all the inner volume of the particle.

22. The particle as claimed in claim 20, wherein the substrate (31) occupies only part of the inner volume of the particle.

23. The particle as claimed in one of claims 20 to 22, wherein the substrate (31) is formed of DNA or RNA.

24. The particle as claimed in one of claims 20 to 23, wherein the substrate (31) is formed of a porous synthetic polymeric matrix.

25. The particle as claimed in one of claims 20 to 24, wherein it contains a liquid composition in its inner volume.

26. The particle as claimed in claim 25, wherein the functional membrane (33) is adapted so as to have a liberation kinetics for the liquid composition following a predetermined profile.

27. The particle as claimed in one of claims 20 to 26, wherein its mean size is between 5 nm and 5 mm.

28. A medicament wherein it includes at least one particle as claimed in one of claims 20 to 27.

29. A supramolecular synthetic film, wherein it comprises a membranous structure as claimed in one of claims 1 to 18.

30. An application for a film as claimed in claim 29 for extracting or separating salts and/or ions from a liquid solution by filtration.

31. A method for preparing a polycationic polymer provided with a plurality of lipidic ligands (65, 66) capable of forming a non-covalent stable lyotropic bond with a stable functional membrane (33, 63) of amphiphilic compounds, so that this polymer can act as a bifunctional compound (32, 62) for fixing the functional membrane (33, 63) on the substrate (31, 61) of a membranous structure as claimed in one of claims 1 to 18, wherein after having carried out the synthetic chemical operations enabling the molecule of the polymer to be obtained, it is put into contact with a citrate in a polar solvent so as to obtain precipitation of the polymer.

32. A polycationic polymer with a purity greater than 95% formed of a polycationic polyamine provided with a plurality of lipidic ligands (65, 66) grafted onto a part of the nitrogen atoms of the amine functional groups, and capable of forming a non-covalent stable lyotropic bond with a stable functional membrane (33, 63) of amphiphilic compounds, this polymer being capable of acting as a bifunctional compound (32, 62) for fixing the functional membrane (33, 63) to the substrate (31, 61) of a membranous structure as claimed in one of claims 1 to 18.

33. The polymer as claimed in claim 32, wherein it has a degree of grafting of the amine functional groups by lipidic ligands of between 1% and 20%.

34. The polymer as claimed in one of claims 32 and 33, wherein it is formed of a succinophospholipidic L-polylysine.

35. A method for preparing a membranous structure as claimed in one of claims 1 to 18, wherein an aqueous suspension is first of all prepared of the bifunctional fixing compounds in the following manner:
a solution is prepared of bifunctional fixing compounds (32, 62) in DMSO,
an aqueous solution is prepared containing at least one non-ionic detergent at a concentration greater than its critical micellar concentration,
the solution of bifunctional fixing compounds (32, 62) is added to the aqueous solution.

36. The method as claimed in claim 35, wherein there is then added to the said aqueous suspension a composition of polyionic polymers capable of forming a solid substrate (33) by polyelectrolytic cross-linking with the polyionic chains of the bifunctional fixing compounds.

37. The method as claimed in claim 36, wherein:
amphiphilic compounds capable of forming a functional membrane (33) are introduced either into the aqueous detergent solution, or into the aqueous suspension before or after adding the composition of polyionic polymers, and so that the concentration of the detergent remains greater than the critical micellar concentration,
the detergent is then removed from the suspension.

38. The method as claimed in claim 35, wherein:
amphiphilic compounds capable of forming a functional membrane (63) are introduced either into the aqueous detergent solution, or into the aqueous suspension,
the concentration of the detergent in the aqueous suspension is then reduced to a concentration less than its critical micellar concentration,
this aqueous suspension is then put into contact with a substrate (61) in the solid phase,
the non-ionic detergent is then removed.
